(19) United States
(12) Patent Application Publication
(10) Pub. No.: US 2008/0069847 A1
(43) Pub. Date: Mar. 20, 2008

(54) Materials containing negative ions and infrared materials for promoting health

(76) Inventors: Young J. Suk, Westbury, NY (US); Lim Sun Hwan, Seoul (KR)

Correspondence Address: WEISS & WEISS, Suite 251, 300 Old Country Road, Mineola, NY, 11501, US

(21) Appl. No.: 11/540,223

(22) Filed: Sep. 29, 2006

Related U.S. Application Data
(63) Continuation-in-part of application No. 11/520,551, filed on Sep. 14, 2006.

(51) Int. Cl. A61F 2/02; A61F 5/00

(52) U.S. Cl. 424/402; 602/1

(57) ABSTRACT

Materials and fabrics compounded with negative ions and infrared materials to promote circulation of blood, ease stress and relieve body pain.

### RELATED APPLICATIONS

The application is a continuation-in-part of application Ser. No. 11/520,551 filed Sep. 13, 2006.

### FIELD OF THE INVENTION

The present invention relates to materials and fabrics compounded with negative ions and infrared materials to promote circulation of blood, ease stress and relieve body pain.

### BACKGROUND OF THE INVENTION

U.S. Pat. No. 4,505,797 relates to electrodyaliss, electrochemical membrane cells and components comprising ion exchange membranes. These membranes are fabricated containing a reinforcing structure comprised of non-woven carbon fibers derived for example, from carbonized polyacrylonitrile. The members so produced have a low d.c. electrical resistance which enables electrodyaliss to be performed more economically.

U.S. Patent publication no. 2003/0208150 relates to a wound dressing or therapy compress containing a film material permanently load with an antimicrobial silver. The dressing or compress comprises a flexible upper backing and a lower shell fastened to one another, each of which comprises a synthetic fabric. The lower shell is fluid permeable and is adapted to be placed in direct or covered contact with a wound or tissue to be treated. The backing may be either fluid permeable or non permeable to allow for the release or retention of moisture. The shell and backing are joined to one another to form a plurality of film material enclosures. The compress may be used to deliver moist heat therapy to an arthritic sprained joint or sprained muscle area.

### SUMMARY OF THE INVENTION

The present invention relates to the use of negative ions and infrared rays. The present invention comprises leather or synthetic fabrics which are compounded with negative ions and infrared materials. The present invention relates to the use of leather or synthetic fabrics which are compounded with negative ions and infrared materials which are then placed around a part of a person's anatomy to help with healing of that part of the anatomy. Positive ions attract atmospheric pollutants, dust particles are harmful dust particles into airspace and keep them there. Negative ions which are abundant in natural places, such as forest, waterfall and the seaside cancel the place of the unnatural environment.

It is an object of the present invention to improve blood flow.

It is an object of the present invention to increase pH levels.

It is an object of the present invention to increase performance in sports.

It is an object of the present invention to increase defenses against infection.

It is an object of the present invention to eliminate pollen, mold spores, dust, pet dander, and other allergy causing particulates from the air.

It is an object of the present invention to freshen the air.

Infrared rays are electromagnetic rays that assist the human body. It is an object of the present invention to use infrared rays to penetrate deep within the body and elevate the body's temperature slightly.

It is an object of the present invention to use infrared rays to increase circulation and metabolism.

It is an object of the present invention to use infrared rays to relieve pain.

It is an object of the present invention for infrared rays to be used to speed healing.

It is an object of the present invention for infrared rays to be used to stimulate organs.

It is an object of the present invention for infrared rays to be used to minimize fatigue.

The present invention relates to a watch, band or collar, placed around a person's wrist or ankles. The band is made of leather or synthetic fabrics and is compounded with negative ions and infrared materials.

It is an object of the present invention for this band to help promote circulation of the blood.

It is an object of the present invention for this band to relieve body pain.

it is an object of the present invention for a belt to be made of leather or synthetic fabrics that is compounded with negative ions or infrared materials and for this belt to be placed around a person's waist area.

It is an object of the present invention for a leather or synthetic fabric that is compounded with negative ions and infrared materials to be placed within and attached to a hat or other covering for the head to be worn on a person's head.

It is an object of the present invention for a hat to be made of leather or synthetic fabrics that is compounded with negative ions or infrared materials and worn on a person's head.

It is an object of the present invention for a knee brace to be made of leather, or synthetic fabrics which are compounded with negative ions or infrared materials and placed around a person's knee.

It is an object of the present invention for an elbow brace to be made of leather, or synthetic fabrics which are compounded with negative ions or infrared materials and placed around a person's elbow.

It is an object of the present invention for a glove to be made of leather or synthetic fabrics that are compounded with negative ions or infrared materials wherein the glove is worn on a person's hand.

It is an object of the present invention for a shoe to have an insert in the shoe which is made of leather or synthetic fabrics and is compounded with negative ions or infrared materials wherein the shoe is worn on a person's foot.

It is an object of the present invention for a heating pad to be made of leather, or synthetic fabrics that are compounded with negative ions or infrared materials wherein the heating pad is worn on different parts of the body.

It is an object of the present invention for a sweatband to be made of leather, or synthetic fabrics that are compounded with negative ions or infrared materials wherein the sweatband is worn on different parts of the body.

The present invention relates to a collar that can be worn by a dog or a cat. The cat is compounded with negative ions and/or infrared materials which promotes circulation blood and relieves body pain for the animals.

The present invention relates to a collar or device that can be worn under the saddle of a horse. Said collar or device being compounded with negative ions and/or infrared materials to promote circulation of blood and relive body pain.

### BRIEF DESCRIPTION OF THE DRAWINGS

FIG. 1 shows the device worn by a person.

FIG. 2 shows the device worn by a dog.

FIG. 3 shows the device worn by a horse.

FIG. 4 illustrates the device worn in a belt.

FIG. 5 illustrates the device in a hat.

FIG. 6 illustrates the device in a knee brace.

FIG. 7 illustrates the device in an elbow brace.

FIG. 8 illustrates the device in a glove.

FIG. 9 illustrates the device in a shoe.

FIG. 10 illustrates the device in a heating pad.

FIG. 11 illustrates the device in a sweatband.

### DETAILED DESCRIPTION OF THE INVENTION

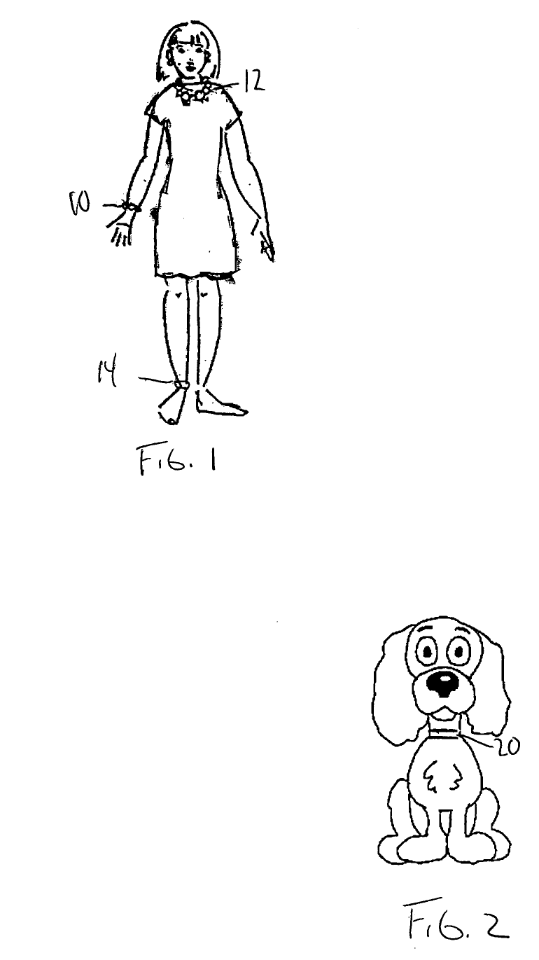

FIG. 1 shows the device 10 worn at or near the person's wrist. The device of the present invention can also be worn as a necklace 12 or as an ankle bracelet 14.

FIG. 2 shows the device 20 of the present invention worn by a dog.

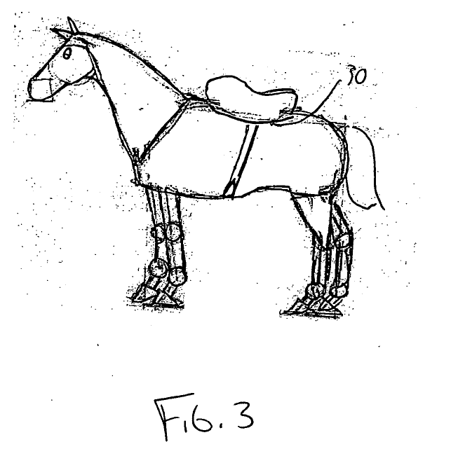

FIG. 3 shows the device 30 worn under the saddle of a horse. The device 30 can also be placed around the horse's legs.

FIG. 4 shows the device 40 being a belt worn around a person's waist.

FIG. 5 shows the device 50 placed in a hat 60 worn on a person's head.

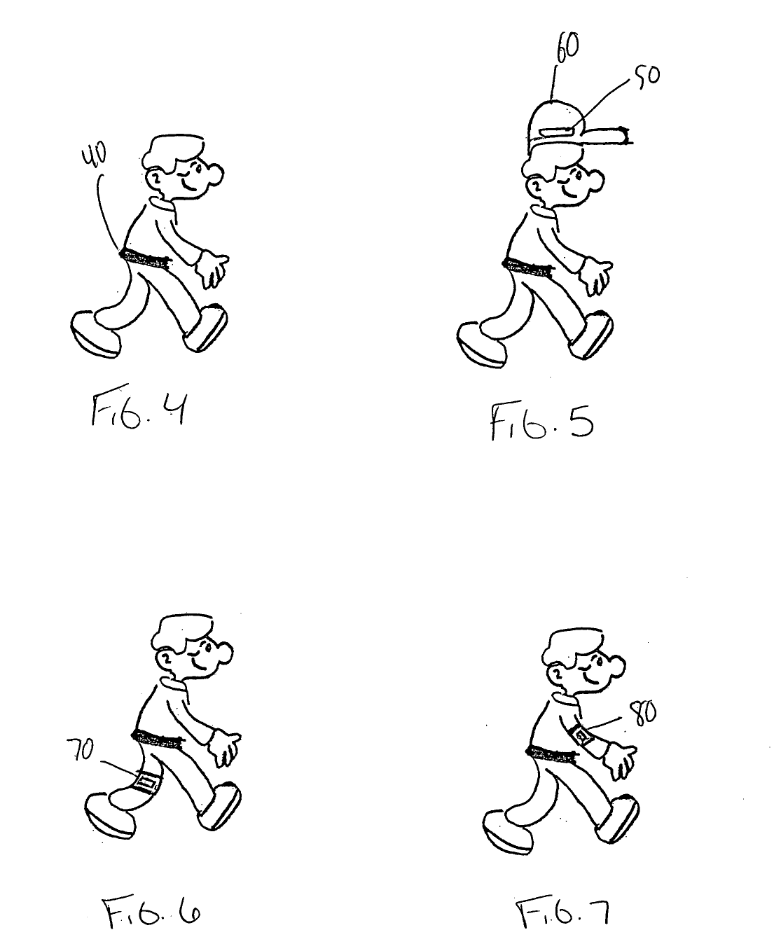

FIG. 6 shows the device 70 being a knee brace worn around a person's knee.

FIG. 7 shows the device 80 being an elbow brace worn around a person's elbow.

FIG. 8 shows the device 90 being a glove worn on a person's hand.

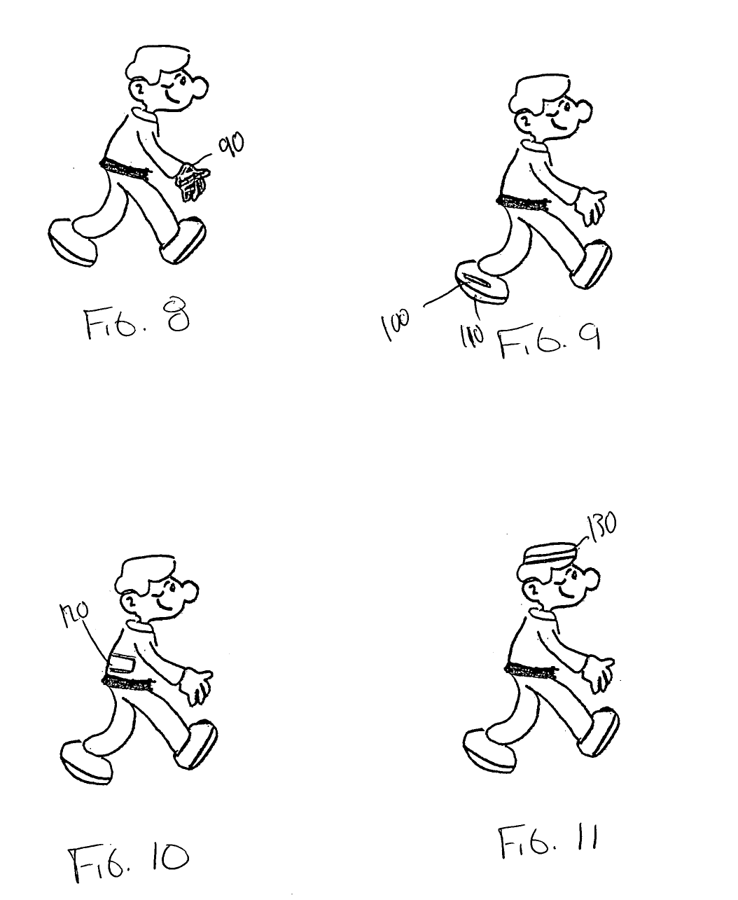

FIG. 9 shows the device 100 inserted into a shoe 110 worn on a person's foot.

FIG. 10 shows the device 120 placed in a heating pad worn on a person's back or other areas needing treatment.

FIG. 11 shows the device 130 in a sweatband worn around a person's forehead.

What is claimed is:

1. A device for promoting circulation of blood, easing stress and relieving body pain comprising;
a leather or synthetic fabric that is compounded with negative ions or infrared materials wherein said device is placed on an area of a person's body.

2. The device of claim 1 wherein said device comprises a belt which is worn around a person's waist area.

3. The device of claim 1 wherein said device is attached to a hat or other covering for the head.

4. The device of claim 1 wherein said device comprises a hat or other covering for a person's head.

5. The device of claim 1 wherein said device is a knee brace.

6. The device of claim 1 wherein said device is an elbow brace.

7. The device of claim 1 wherein said device is a glove.

8. The device of claim 1 wherein said device is an insert for a shoe.

9. The device of claim 1 wherein said device is a heating pad.

10. The device of claim 1 wherein said device is a sweatband.
